(19) United States
(12) Patent Application Publication
(10) Pub. No.: US 2017/0010223 A1
(43) Pub. Date: Jan. 12, 2017

(54) SYSTEM FOR MOLECULAR IMAGING

(71) Applicant: Tumay O. Tumer, Beverly Hills, CA (US)

(72) Inventor: Tumay O. Tumer, Beverly Hills, CA (US)

(21) Appl. No.: 15/274,690

(22) Filed: Sep. 23, 2016

Related U.S. Application Data
(63) Continuation-in-part of application No. 10/557,016, filed on Nov. 16, 2005.
(60) Provisional application No. 60/479,849, filed on Jun. 20, 2003.

(51) Int. Cl. G01N 23/04; G01N 33/60; A61B 6/00; G01N 33/50

(52) U.S. Cl. CPC G01N 23/04; G01N 33/5008; G01N 33/60; A61B 6/481; B82Y 35/00

(57) ABSTRACT

Charged and neutral particles, photons (13), photonic optics, detectors (15) and sensor arrays are used for application to molecular imaging, communication with biological organisms and monitoring and learning biological activity inside living organisms. The living organisms include among others living tissue, biological organs, cells (10), eukaryotes, prokaryotes, viruses and phages. Molecular imaging can be an effective new tool to understand the mechanisms of life and communicate, modify and control it. Techniques, methods and devices are described to achieve these aims. The probes used in molecular imaging described above will include the full spectrum of photons; charged and uncharged particles (13); chemicals; and biological probes.

### CROSS REFERENCE TO RELATED APPLICATIONS

This application is a continuation-in-part of U.S. patent application Ser. No. 10/557,016, entitled “SYSTEM FOR MOLECULAR IMAGING,” filed on Nov. 16, 2016, by Tumay O. Tumer;

U.S. patent application Ser. No. 10/557,016 is the 371 national phase filing of Patent Cooperation Treaty Application Number PCT/US04/19233, entitled “SYSTEM FOR MOLECULAR IMAGING,” filed on Jun. 18, 2004, by Tumay O. Tumer;

Patent Cooperation Treaty Application Number PCT/US04/19233 claims priority benefit of U.S. Provisional Patent Application Ser. No. 60/479,849, entitled “SYSTEM FOR MOLECULAR IMAGING,” filed on Jun. 20, 2003, by Tumay O. Tumer, and all of the above listed applications are incorporated herein, by reference, in their entirety.

### GOVERNMENT RIGHTS NOTICE

This invention is not made under U.S. Government grants. The U.S. Government has no rights on this invention.

### FIELD OF THE INVENTION

The invention described uses charged and neutral particles, photons, photonic optics for all wavelengths, detectors and sensor arrays for application to molecular imaging and communication with biological organisms and biological activity inside living organisms. The living organisms include among others living tissue, multicellular organisms, monocellular organisms (solitary cells or protists), biological organs, cells, eukaryotes, prokaryotes, viruses, phages, prions, etc. (Cell is used here to mean any or all types of cells including but not limited to eucarya, eubacteria, archea, eukaryotes, prokaryotes, viruses and phages).

Molecular imaging here is used in a larger and more general sense such as visualizing, studying, learning, understanding, communicating, modifying, producing, governing and controlling living organisms and life. Therefore, molecular imaging can be an effective new tool to understand the mechanisms of life and communicate, modify and control it. This invention will describe the techniques, methods and devices to achieve these aims. It will help in understanding the biological mechanisms driving life and will result in understanding, learning, communicating, controlling, curing, preventing, eliminating, creating, producing chemicals and drugs, improving and enhancing biological processes and life, and preventing, controlling and curing diseases. The probes used in molecular imaging described above will include the full spectrum of photons from radio waves to gamma rays; charged and uncharged particles such as electrons, positrons, protons, antiprotons, neutrons, muons, pions; chemicals; and biological probes such as living organisms, cells, prokaryotes, viruses, phages and prions. The living organisms and cells are observed and imaged using detectors, imaging sensor arrays, and all types of microscopes. Molecular imaging requires high spatial resolution detectors and sensor arrays with resolution approaching and surpassing the dimensions of biological organisms and molecules. Magnification may also be required. High-energy resolution is important to get full spectral information with great detail, 10% to less than 0.01% FWHM (Full Width Half Maximum), for most of the energy range. Energy spectrum observed will range from radio waves to gamma rays. A wide range of particles may also be used for probing, emission and imaging, such as photons, electrons, positrons, protons, antiprotons, neutrons, muons, pions, etc. Biological probes both in the form of living organisms and in chemical form may be used. Magnification of the image, signal, photons, rays and radiation will be applied and used to increase spatial resolution in identifying the molecules, cluster of molecules and biological features and components of living organisms under study. Stereoscopic, two dimensional (2D), tomographic, three-dimensional (3D) and holographic imaging will be carried out to produce two or three-dimensional images. Wireless data transmission from the measurement, probing and imaging site to internal or external data acquisition system will be undertaken. This will be achieved by using microwaves to radio waves, JR, UV and optical emissions and transmissions. Special chemical markers and radiopharmaceutical will be used to tag and follow molecules and molecular groups and/or clusters. One or more cells or living organisms may be developed with radiation tagged molecules and atoms and the emitted radiation is imaged using high-resolution detectors and sensor arrays with or without image magnification. The different sensors, detectors, probes, technologies and methods can be integrated to improve imaging and measurements. Using these instruments and technologies in combination may improve measurements, data acquisition and understanding of the molecular and biological activity.

### BACKGROUND OF THE INVENTION

Imaging molecular activity in biological processes and life depends on new understanding of the field. Life by itself is very complex and its origin, reason, diversity and the underlying mechanism are not yet fully understood. Evolution has been the most successful theory to explain the development of life. However, it has shortcomings in explaining its diversity, increase in complexity with time and sudden immergence of species such as Cambrian Explosion. Cambrian explosion remains unexplained since its discovery. During this period single eukaryotic cells proliferated or transformed into multicellular organisms with hard body parts such as shells and skeletons about 540 million years ago (Smith and Szathmary, 1999). These hard body parts produced the fossilized evidence, which documents this historic event. There are body impressions of soft-body multi-cellular organisms called Ediacara discovered in the fossilized mud just before this period, at the end of Precambrian era (Fortey 1999). Ediacara organisms failed to survive Precambrian and became completely extinct, never to reappear again. To our knowledge, for the first time such a complete extinction has happened as if the Ediacara could not protect itself from the new species just emerging or may be this body form was abandoned. At the end of Precambrian and at the beginning of the Cambrian periods a great transformation has happened where single eukaryotic cells formed multicellular organisms. Cambrian explosion is contrary to the slow progress of evolution and cannot be explained convincingly using the theory of evolution. Therefore, it is an important clue in understanding the underlying mechanism of the theory of evolution and perhaps the origin of life itself.

Natural selection is not considered as the sole source for the evolution of new species and mutation is generally accepted as an important contributor to the emergence of new species. However, to produce a new species numerous mutations have to happen in the parent simultaneously or almost simultaneously so that a working new species, which can live and sustain life can evolve. The mutation(s) must happen in the cell especially in the genetic code or DNA. However, most mutations in DNA can destroy its cell which means that most mutations does not lead to evolution, especially if the mutations are random in nature. Therefore, to produce such new complex life forms and new species or phyla, the number of random mutations required are statistically astounding unless the mutations are somehow biased in the right direction that is leading to a better organism or one that fits better to the conditions or environment. There is no known direct evidence that vast number of random mutations are happening in the present population or it has happened in the past. Natural selection is too slow to give rise to new species in a relatively short time. There is, in fact, significant evidence that new species are arising in relatively short time compared to evolution (Gould and Eldredge 1972). Therefore, some scientists feel the evolution, or natural selection, alone cannot explain the emergence of new species and also the Cambrian explosion (Eldredge 1995; Goodwin 1994; and Wesson 1991). Random mutations are also not an acceptable alternative. Only possibility is biased mutations in the direction of the emergence of new species that fit their environment and conditions better. Therefore, there is an important clue in the production of new species in understanding the origins of life, how it is continuously evolving into more and more complex life forms and its underlying mechanism.

Although, evolution is very well established and extremely successful there is no widely accepted explanation yet on how it works and what are the underlying driving mechanism(s). There are several major discovered events that provide strong evidence for understanding the mechanism of evolution and how it may be working. Examples for these are the Cambrian explosion, Permian extinction, increase in complexity with time and the accelerated emergence of some species. Therefore, it may be necessary to consider these and emergence of new species under a different light. Of course, the first and most relevant event, which is providing the most profound evidence, is the Cambrian explosion. If no other laws of nature were acting other than the natural selection then the emergence of multicellular organisms would have been a gradual process with variety of forms also increasing slowly in time. However, the relatively sudden emergence of multicellular organisms with vast variety of different forms is showing that the underlying mechanism of evolution may be quite different than what is considered. The most widely accepted mechanism of evolution today is mutation in the DNA. Mutation is a blind random process and is most often fatal to the cell (Alberts et. al, 2002). Some mutations are silent, do not produce any effect and a few produce beneficial results which leads to evolution. Rate of mutations are extremely slow otherwise it would be impossible to maintain life we know today. Therefore, the DNA is conserved and stable and replicates extremely accurately. It has been determined approximately that the rate of mutation is roughly 1 nucleotide change per 109 nucleotides each time that DNA is replicated (Alberts et. al, 2002), which is approximately the same for the bacteria and the human cells, which is surprising. The rate of change is also measured in humans where the sequence comparisons of the fibrinopeptides indicate that a typical protein 400 amino acids long would be randomly altered by an amino acid change in the germ line roughly once every 200,000 years (Alberts et. al, 2002). This demonstrates the slow process for evolution. Therefore, to create new species and phyla in such short time, as seen during the Cambrian explosion, an astounding number of mutations are required in a relatively short time. Any mechanism in the cells that can do such a feat is not yet seen or discovered. Therefore, to produce a functioning new and complex multicellular living organism, a new, yet unknown, process and/or mechanism is required which can produce large number of mutations biased in the direction to develop the new species in relatively short time. If there is such a biasing mechanism for the mutations then evolution of new species can be a rapid process as seen during Cambrian explosion. This alone is not sufficient to explain the large variety of new complex organisms formed during the Cambrian explosion. However, this fact may be an important evidence on the nature of this biasing mechanism itself.

After the Permian extinction one would have expected a Cambrian type explosion if some of the present explanations of the Cambrian explosion are correct. However, there was no such explosion in the variety of organisms and no new phyla appeared. This can be another important evidence to consider and study in understanding the mechanism of evolution of new species and phyla.

### PRIOR ART REFERENCES

The following are prior art references for this application:
1. Dyson, Freeman “Origins of Life” Second Edition Cambridge University Press (1999).
2. Eldredge, Niles “Reinventing Darwin” New York: John Wiley (1995).
3. Fry, Iris “The Emergence of Life on Earth: A Historical and Scientific Overview” Rutgers University Press (2000).
4. Goodwin, Brian “How Leopard Changes Its Spots” New York: Scribners (1994).
5. Gould, Stephen Jay and Eldredge, Niles “Models in Paleobiology: Punctuated. Equilibrium: An Alternative to Phyletic Gradualism” (1972).
6. Gould, Stephen Jay “Wonderful Life” New York: Norton (1989).
7. Kauffman, Stuart “Investigations” Oxford University Press (2000).
8. Morris, Richard “Artificial Worlds: Computers, Complexity and the Riddle of Life.” Plenum Trade, a division of Plenum Publishing Corporation (1999).
9. Schlesinger, Allen B. “Explaining Life” McGraw-Hill Inc. (1994).
10. Smith, John Maynard & Szathmary, Eors, “The Origins of Life” Oxford University Press, (1999).
11. Wesson, Robert “Beyond Natural Selection” Cambridge, Mass.: The MIT Press (1991).
12. Fortey, Richard A. “Life: a natural history of the first four billion years of life on earth.” Vintage Books, (1999).
13. Darwin, Charles, “The Illustrated Origin of Species” Abridged & Introduced by R. E. Leakey. Hill and Wang (1979).
14. Alberts, B. et al., “Molecular Biology of the Cell,” Fourth Edition Published by Garland Science, (2002).
15. Pollard, T. D. & Earnshaw, W. C., “Cell Biology,” Revised Reprint by Saunders, (2004).
16. Mayr, Ernst, “What Evolution Is,” Basic Books (2001).
17. Watson, James D., “DNA: The Secret of Life,” Knopf, Borzoi Books, (2003).

### DISCLOSURE OF INVENTION

If all the factors listed above are considered, then intelligence embedded into the cells emerges as the most plausible mechanism and candidate which may be driving evolution. What is meant by intelligence is that the cells forming the multicellular organisms may have developed intelligence sufficient to accomplish such a feat during their billions of years of evolution before the Cambrian period. Although, attributing intelligence to tiny cells may be considered improbable, however, if it is the case, it explains well the Cambrian explosion with proliferation of variety of multicellular life forms and the accelerated evolution of some species. It also provides the mechanism for increasing complexity with time, major organism adaptations such as moving onto land, flying, seeing, acclimatization, etc.

If single cell organisms have developed intelligence before the Cambrian period, then the Cambrian explosion can be explained as the time when the cells have discovered how to form multicellular organisms and they applied their discoveries to form the variety of different multicellular life forms. This may be considered as experimentation and may explain why so many different species have been formed in such a short time after billions of years of evolution of single or solitary cell organisms. It also explains why the number of species continued after Cambrian Explosion is much smaller, as the successful models were naturally selected to move ahead and the failed experiments could not compete or abandoned. This is similar to the proliferation of experiments humans are carrying out during the past 5,000 years through their intelligence, which may be called the start of the Quaternary or more correctly Holocene explosion.

The cells that form the multicellular organisms are eukaryotes. Where this intelligence for eukaryotes emerged from is important for the theory of the origins of life. The other cells that are well known are the much smaller prokaryotes (microbes), viruses and phages. These cells are not known to produce multicellular organisms. However, they could be the progenitor of eukaryotes. Therefore, prokaryotes must have had intelligence in order to discover and evolve into eukaryotes. However, their intelligence is likely more elementary than eukaryotes as it is evident from what eukaryotes have accomplished. It is therefore interesting to search and understand if prokaryotes have lived before eukaryotes. This is difficult as without skeleton and small size prokaryotes and eukaryotes do not easily leave behind fossil records. However, it is important to understand the connection. The next known intelligence level can be viruses and phages. It is even more difficult to find, study and understand if viruses are progenitor of prokaryotes. However, this may be a potentially good possibility and may be studied in laboratory by accelerating evolutionary process or mechanism(s). Therefore, in this way new eukaryotes can be engineered to evolve from prokaryotes. Similarly, new prokaryotes can be engineered from viruses and phages. Another known link near the bottom of the intelligence chain can be the prions. They are not a cell. They are large protein molecules that can duplicate and infect their host. Therefore, the life and may be the intelligence of prions are even at a more elementary level. Again it may be possible to propose that prions could be the progenitors of phages or viruses but this is a remote possibility.

The intelligence among eukaryotes also may be different from one species to the next such as the eukaryotes of humans and mouse, for example. On the other hand, it is plausible that the fundamental intelligence in the eukaryotes may be the same between the two cells but the code or the blue print developed in the form of RNA/DNA may be the reason for the difference in the level of intelligence between the species in the evolved multicellular organisms.

Other than cells, phages and prions there is no known or discovered demonstration of intelligence by any other known entity, excluding multicellular organisms. Even attributing intelligence to viruses, phages and prions is difficult as their life looks robotic in nature or preprogrammed. Their actions may be guided under simple intelligence and not robotic since they mutate and develop immunity to drugs which may be considered intelligence in nature and not robotic or preprogrammed. The origins of prions may be attributed to chemical processes or may be to even more elementary intelligence. Therefore, life may have evolved from the most elementary intelligence present in the universe to the present level on Earth or even to higher levels elsewhere in the universe. It is important to study, understand and discover the intelligence and its nature in the world. The invention described here in combination with or without intelligence in life forms and living organisms can lead to the development of methods, instruments or apparatus such as described here that can be used to learn, understand, study and communicate with this intelligence. These methods, devices, instruments and apparatus may also be used to evolve life into new forms, develop them into probes and make biological and intelligent instruments, devices and apparatus.

Different embodiments of this invention will depend on and make use of the intelligence in living organisms in nature. The intelligence in living eukaryotes is likely to be higher than the intelligence they have created in the multicellular organisms. This is generally true except for humans. Humans may reach or surpass the intelligence of their cells, eukaryotes, one day. This may be facilitated by utilizing the invention described here.

Different embodiments of this invention can be used to search, measure, find, learn, understand the nature of the different levels of intelligence existing in the universe. Positive identification of such intelligence may mean a new definition of life is necessary. Definition of life has been a major problem as there is no explanation that defines every living being encountered. Reproduction is thought to be the most fundamental definition of life at present as noted by many scientists. The explanation above leads us to a totally different and unique definition of life, which is intelligence. Intelligence may also be the underlying mechanisms of evolution. If proven and demonstrated to be correct then only one unique identifier, intelligence, will define and become the basic definition for all life and life forms throughout the universe.

This may also mean that the life has emerged from the most elementary level of intelligence and grew into very complex form on Earth that we know today. This is similar to the extremely simple and basic logic in computers electronics can produce ultra complex electronics devices and programs. It also means that the most fundamental level of intelligence in the Universe is not known yet and must be searched and discovered or proven not to be the case. The explanation is likely lying in physics and/or chemistry, which is the law of nature in the Universe. Therefore, the fundamental intelligence in the Universe may also be a law of nature.

The complexity on Earth is evolving and increasing continuously. A natural consequence of intelligence is growth of complexity with the increase in time. That is, intelligence leads to complexity. This may explain an important observation in nature, evolution of more and more complex life forms in time, which was not satisfactorily explained yet by the Theory of Evolution. Therefore, intelligence, if it is the underlying mechanism of evolution, may explain this difficulty in this amazing and successful theory.

The invention in its many embodiments described here can be a foundation to search for the fundamental intelligence in the Universe and its level of increasing complexity. This is because, if such fundamental intelligence exists in the universe in ever increasing complexity it must be present in all the living beings we observe from prions, phages to eukaryotes at some level. Once this intelligence is understood and discovered using the different embodiments of the invention described here, then this information can be used to build intelligent life forms, tools, instruments and apparatus, also described here. Therefore, the best way to discover or disprove this intelligence is to observe and study the living organisms from prions to eukaryotes. Since eukaryotes are the most intelligent of them all, as they have created the multicellular organisms and humans, and the largest in size they are the most important to study. Present techniques only allow a study of gross structure of the eukaryotes and prokaryotes. Therefore, a much finer and detailed imaging of the biological and chemical activity within the inner structure of the cells at molecular level is required to study the cellular intelligence. High spatial and energy resolution imaging detectors are used with or without magnification to look at the dynamic or metabolic life and biological function of an individual or groups of cells.

The intelligence in the Universe is expected to be everywhere and it will develop complexity and complex life forms if the environmental conditions are not hostile for life and stable for long periods of time. At any part of the Universe if the environmental conditions are favorable the intelligent component can form life starting at its basic level and advance from there to build complexity. The level of intelligence that will form and how fast it will advance what level it will reach will be only limited by the hostility of the environmental conditions and the allowed time where the favorable condition will be stable. If the environmental condition worsens it may adversely affect the formation and advancement of the complex life Also, the complexity of life and level of intelligence may be synonymous.

The different embodiments of the invention described here for Molecular Imaging discover the cellular intelligence then this can open new fields of study and also can lead to new techniques, methods, probes, life forms, apparatus, instruments for molecular imaging such as described here. These can also be used for medical procedures; disease prevention, control and cure; treatment methods; and drug discovery, testing and verification, etc.

We see only the gross structures in a cell, which is about 10-100 micrometers diameter. Radius of a Hydrogen and a Carbon atom is 25 and 77 picometers, respectively. Therefore, eukaryotes contain approximately 1016 to 1018 atoms depending on its size. In comparison, human body has roughly 1013 cells. Such high number of atoms eukaryotes contain show that although cells are very small they have the ingredients they need to form the complexity which may have formed the intelligence.

Some further evidence for cellular intelligence in addition to what is described earlier are the motion, chemical processes and biological activity observed inside the cells such as cell division and self repair shows that all these actions happen under complete control and not a random process such as Brownian Motion. Therefore, such behavior is more symbiotic with intelligence than preprogrammed or robotic in nature.

Another factor is the number of genes a cell contains, which is attributed to the life and evolution. It is not possible to produce the complexity a human being has with about 30,000 genes in their chromosomes. Some scientists believe the number may be as high as 100,000. However, even 100,000 genes cannot produce a human being with the complexity it has. Chromosomes, of course, contain much more information and data, which is thought to be mostly redundant, repeats (about 53% (Watson, 2003<< and not used, but sufficient to produce such a complex being. The number of genes goes from about 1,000-4,000 (1,590 for Helicobacter Pylori) in bacteria to about 30,000 in humans. This is about a factor of 7-20 larger in the number of genes but there is vast difference in complexity between bacteria and humans. We know that the human chromosomes contain a much larger amount of information then the chromosomes of bacteria in accordance with the increase in complexity. For example, genome size (measured in 1000s of nucleotide pairs per haploid genome) for Helicobacter Pylori is about the same size as its number of genes, (1,667 for Helicobacter Pylori) (Alberts et. al, 2002) about 5% larger, but the human genome size is 3,200,000 (Alberts et. al, 2002) over 10,000% larger than its number of genes. Therefore, the human complexity difference compared to the bacteria may be most likely residing in what appears to be in the vast redundant and unused parts of the human DNA, the blueprint of human beings, unless there is another source for the data to produce multicellular organisms such as human beings. The reason for considering this vast database as unused may be because the biological activity of a cell cannot be observed and studied closely and the “non genetic” section of the DNA, therefore, has not yet been seen in action. The genes and the biological activity detected by the scientists may be the tip of the iceberg because only the gross or large scale biological processes and activity can be observed using present technology leaving behind a vast majority still waiting to be discovered. This is why this invention is important, so that scientists can probe deeper and with higher resolution into the cellular life using the devices, instruments, methods, etc. described here to study, learn and discover the remaining secrets of cellular life, which may discover and demonstrate the cellular intelligence. What we learn can then be utilized to increase complexity and may be advance life further into unprecedented new levels.

The way to accomplish this is either magnify the image to the level where we can observe it clearly with detail (FIG. 4), or send probes into the cell, such as molecules, bacteria, viruses and phages with defined targets and functions and learn from them the processes going on inside the cell. This may also be called to reduce our vision to the size of the cell. That is to look inside a cell using intermediary eyes. The molecules may also be specially engineered compounds such as proteins, enzymes, RNA, DNA, and their fragments. The probes may also be tagged by a fluorescent die(s) or a radioactive atom(s) (nuclei). Therefore, their path and actions can be tracked and observed by imaging the emitted fluorescence photons either naturally or through simulation by laser or other means, or by detecting and imaging the emitted particles such as positrons, alpha particles, x-ray and gamma-ray photons emitted by the radioactive agent(s). The emission of fluorescence photons and particles are random in direction and isotropic. Therefore, a high magnification focusing or imaging system is required. To image fluorescence radiation a high magnification microscope system can be used. The spatial resolution of the images can be improved by using technique such as interference and phase contrast imaging.

Imaging particle emissions from tagged radioactive nuclei can be done differently depending on the emitted particle. If charged particles are emitted such as beta, alpha and positron, they can be imaged using an electromagnetic focusing system such as a solenoidal or other type quadrupole focusing magnet. Although positrons will annihilate if they meet an electron, since the cells are so small most of these particles will come out without annihilating or losing much energy. If the charged particle or positron focusing system is in vacuum or near vacuum or low electron count gasses at low concentrations are used such as hydrogen and helium then the charged particles and especially positrons can be focused and imaged with high resolution. These particles may also be accelerated during imaging if necessary using electric fields. On the other hand, if the emitted particle is a photon 90 then a different kind of focusing system may be used such as Bragg reflection mirrors for low energy x-ray photons or capillary focusing systems. It is also possible not to use a focusing system but use nanotechnology to develop detectors 91 of the size of cell(s) (FIG. 9). To image a cell with size of about 10-100 micrometer the pixel detector (FIG. 6) will require pixel size or pitch in the range of about 01.×0.1 to 10×10 micrometer. The imaging detector will also need a collimator 92 developed by nanotechnology of similar dimension holes to produce an image if the detector is at a distance from the cell(s). If the detector is in contact or near contact distance then the collimator may be omitted or made thin or made with larger size holes. This technique will be good to image low energy x-rays 90 and the charged particles 90 emitted by tagged molecules in the cell. The pixel detector will also require an integrated circuit (IC) 93 or Application Specific Integrated Circuit (ASIC) to read out the detector as shown in FIG. 6. Since the pixel pitch is very small it will be necessary to use foundry processes with ultra thin line or gate width less than 0.35 micrometer. There are processes already available which have minimum line widths of 0.09 micrometer. Development of processes with even smaller line widths are under development. The detector array also needs to be connected to the readout chip. This can be done in several ways. One embodiment is to deposit the detector material right onto the readout IC. A second method is to use indium bump bonding 94 or other bump bonding 94 techniques for flip chip processing to mount the detector onto the readout IC. Therefore, with the new technologies presently available a cell size pixel detector can be designed and fabricated. This detector will be used to image a single cell 10 or a group of cells. 10. The cell nucleus 11 will be also imaged. It can also be used to image other cellular organisms such as bacteria or tiny objects, devices or instruments such as nanotechnology products.

### SPECIFICATIONS AND DIFFERENT EMBODIMENTS

Imaging molecular activity, biological processes and understanding the life and the intelligence producing the life can lead to following fields, instruments, devices, methods, techniques, probes, apparatus and imaging systems.

1. Make imaging systems to produce two-dimensional (2D), three-dimensional (3D), tomographic, holographic and/or stereoscopic images of the cellular structure; biological structure, functions, and activity; chemical structure and activity; the nature, components and form of its intelligence of all living organisms. The 3D, stereoscopic and tomographic imaging can be achieved by using two or more imaging detectors such as shown in FIG. 6 and FIG. 9 in required configurations to produce the desired images. In tomographic imaging the detectors can be rotated around the cell(s) or formed as a ring or cylinder to surround the cell(s).

2. Wireless data transmission from the measurement, probing and imaging site to internal or external data acquisition system will be undertaken. This will be achieved by using microwaves to radio waves such as Blue Tooth technology, IR, UV and optical emissions and transmissions. Special chemical markers and radiopharmaceutical will be used to tag and follow molecules and molecular groups and/or clusters. The different sensors and the methods can be integrated to make measurements and imaging using them in combination to improve edata acquisition and understanding of the molecular activity.

3. Develop large magnifying imaging systems to observe the cells in much higher resolution and detail than available at present. This can be achieved by producing an x-ray 13 or gamma ray 13 beam from an ultra small focus 14 or source 24. With present technology it is possible to produce micro (10⁻⁶ m) (such as Tosmicron x-ray sources by Toshiba, Luminous by Pony Industry and XTG Microfocus X-ray Source by Oxford Instruments) or even nano (10⁻⁹ m) source or focus size x-rays (FIG. 1 and FIG. 2). Nano focus x-ray sources are doable because to image a cell low flux x-rays needed at lower energies. With such a small source point size the x-rays going through the cell(s) can be magnified highly to produce a high resolution image of one or more cells. The detector 15 can be made in many different ways. For low energy x-rays the best detectors can be silicon pixel or strip detectors. One can also use other type of pixel or (single or double sided) strip detectors such as GaAs, Diamond, Selenium, CdZnTe, CdTe, Hgh, Pbh, etc. The detector array can be made by just a single pixel or strip detector or by tiling two or more detectors as shown in FIG. 7.

4. Use robotic or intelligent probes that will provide information about the inner workings or chemical, physical and biological structure and activity inside the cells. This will be achieved by making molecular probes such as chemicals, proteins, enzymes, RNA and DNA molecules and sections. One can also develop intelligent probes. This can be done by using bacteria, viruses, or phages, which are genetically programmed to be used as probes to learn and image inside a cell and monitor its activity.

5. Reduce our vision to the size of the cells, that is to make detectors about the size of cell(s) (FIG. 9) like an eye to view from outside or even inside a cell the activity and structure of the cell or living organisms.

6. Make probes or devices that can disrupt and/or disturb a process inside the cell and provide information on the reaction of the cell. This may be an interesting way to detect and demonstrate intelligence in the cell. It would be an invaluable experiment if a disturbance, which cannot have been preprogrammed, can be made and the cell can respond to it in time in an intelligent manner.

7. Make imaging systems where the photon source is generated from micro, nano or pico focus or source size, with focus or origin of the photon source is 10⁻⁶ to 10−¹⁵ m diameter. The photon or particle beam diverging from such a fine focal point (FIG. 1 and FIG. 2) will allow high magnification to produce high resolution and detailed view of a cell, a life form or a living organism at different wavelengths.

8. Make an imaging system where a parallel beam of photons or particles are used to image a cell or living organisms. The outgoing or emerging beam from the object is then diverged inside or under the action of a system to produce a magnified image (FIG. 3). This can be achieved by using a parallel beam of charged particles 34 such as electrons 13 or protons 13. The particle beam after passing through the cell(s) 10 diverged by using electro magnets 35 such as dipole magnets to form a large image. The image is detected and recorded by a position sensitive detector 15. Pixel detectors (FIG. 6) and arrays (FIG. 7) can be used to produce the image. It is also possible to make the image without diverging the beam by using small pixel detectors about the size of the cell(s) (FIG. 9). In this case the collimator 92 may not be needed.

9. Include and/or tag chemicals and molecules inside a cell, a life form or a living organism with material that emits radiation or particles. The radiation can be of any type such as photons of any wavelength including but not limited to visible, IR, UV, x-rays, florescence, gamma rays, microwave, and radio waves. Particles can be neutral or charged, which include but not limited to electrons, positrons, protons and alpha particles.

10. The detectors (FIG. 5 and FIG. 6) and sensor arrays (FIG. 7) used to produce the image can be made planar, spherical or cylindrical form for uniformity and high accuracy without artifacts. TFT detector arrays may also be used.

11. Make a miniaturized high resolution system for Magnetic Resonance Imaging (MR!), MRI Spectroscopy and/or Functional MRI and image and study single or multiple cells, life forms and living organisms as a whole or its parts. Tune some of these devices to focus on a certain atom, nuclei or molecule. Make these instruments using nanotechnology to achieve compact size and the high resolution.

12. Use and/or make high resolution imaging instruments using phase contrast, bright field, dark field, interference, polarization, differential interference contrast, fluorescence, cell electrophoresis, hydrodynamics, NMR, transmission and standard electron microscopy, transmission and standard proton microscopy, crystallography, confocal microscopy, laser probed imaging, to image and study cell(s).

13. To create phages or bacteria with probe(s) inside in the form of DNA or other molecules and use them to inject or transport the probe into cells to learn about the cellular activity, biology, chemistry and physics.

14. Establish and conduct communication with cells and living organisms including but not limited to eukaryotes, prokaryotes, viruses, and phages, using electronic, chemical, physical or biological techniques and methods to form new life forms, biological instruments, probes, devices. This method(s) and instrument(s) may be also used for medicine and health care such as to cure, control or prevent diseases. The communication with the cells will be achieved by first learning and understanding their intelligence and how it works using the methods and the technology presented. It is also important to learn cell-to-cell communication. After learning cellular intelligence and how the cell-to-cell communication is conducted then a communication will be devised to communicate with cell(s) especially if the cell is conscious.

15. Study, learn and make use of communication between cells.

16. Establishing contact with cell(s) either through unintelligent and/or intelligent way can be made from outside or inside the cell. The communication mayor may not affect or disrupt the cellular activity. A door into the cell may need to be created to establish contact inside the cell which does not kill or grossly disrupt the cell. The door(s) may be produced in different ways including but not limited to the following methods and techniques to create door(s) or gate(s); using phage(s) or bacteria; nano technology; nano tubes; chemical techniques; and physical or nano technology. External communication can be established using these techniques. For example, using phages may be the best way to go. This because phages naturally produce a door into the cell's membrane to inject its DNA into the cell. Similarly, phages can be used to open door(s) into cells, send probes in and communicate with cells. Phages mainly attack bacteria, therefore, it may need the development of new phage type systems which can open doors into eukaryotes. It is essential that these phage type organisms thus created must not be able to carry out self duplication as they may become a new infective agent to humans.

17. Communicate, supply information and data, and enhance the cellular intelligence.

18. Receive information and data from the cell(s) learn and understand the cellular intelligence and use this knowledge to create new life forms; instruments; apparatus; methods; techniques; structures; and prevent, control and cure diseases.

19. Improve and help the advancement of human and all animal and plant life using the knowledge gained.

20. Secure future of life on earth, initiate quick recovery or prevent mass extinctions, so that evolution can proceed rapidly and unhindered.

21. Develop and produce new food sources by genetically engineering living tissue cultures that can grow rapidly at low cost.

22. Control and reduce waste generation. This will be done by producing food that will produce little waste products, that is it will be totally digested by human beings and animals.

23. Improve tolerance and adaptation of life to different, changing and challenging environments and changes in environment and ecosystem. This will be achieved by communicating the problem to the cell and helping cell reprogram its genome to produce quick adaptability. Also it will allow correcting the ecosystem problems by developing new generations, cloning, adaptability, new specific life forms, etc. Trying to help ecosystem can be very dangerous and must be carried out very carefully. Otherwise more harm may be done.

24. All these must be very carefully controlled by the Government so that harmful and disastrous actions against humanity and all other living beings cannot be undertaken by people or organizations using the new technology.

25. Solve environmental problems including but not limited to pollution; ecosystem losses and changes; and habitat improvements.

26. Learn and teach the technology, science and engineering developed, created and accumulated by the cells.

27. Engineer and produce new drugs using cells and cellular intelligence.

28. Develop new fields of study such as nature, foundation and advancement of intelligence in Universe; cellular intelligence; communication with cells; cell engineering; high resolution molecular imaging; bio-computation; cellular pharmacology; formation of new life forms; developing biological instruments; etc. These will be achieved by communicating with cell(s) and imaging cellular activity.

29. Produce intelligent nanotechnology using cellular or related intelligence. Develop intelligent nanotechnology or bionanotechnology by developing nanotechnology instruments, apparatus, devices, etc. using intelligence learned from cells and/or with biological form and components.

### BRIEF DESCRIPTION OF THE DRAWINGS

FIG. 1 is a block diagram of the molecular imaging instrument imaging cell(s) using a focusing into a small vertex then diverging beam of photons or particles to magnify and image cell(s).

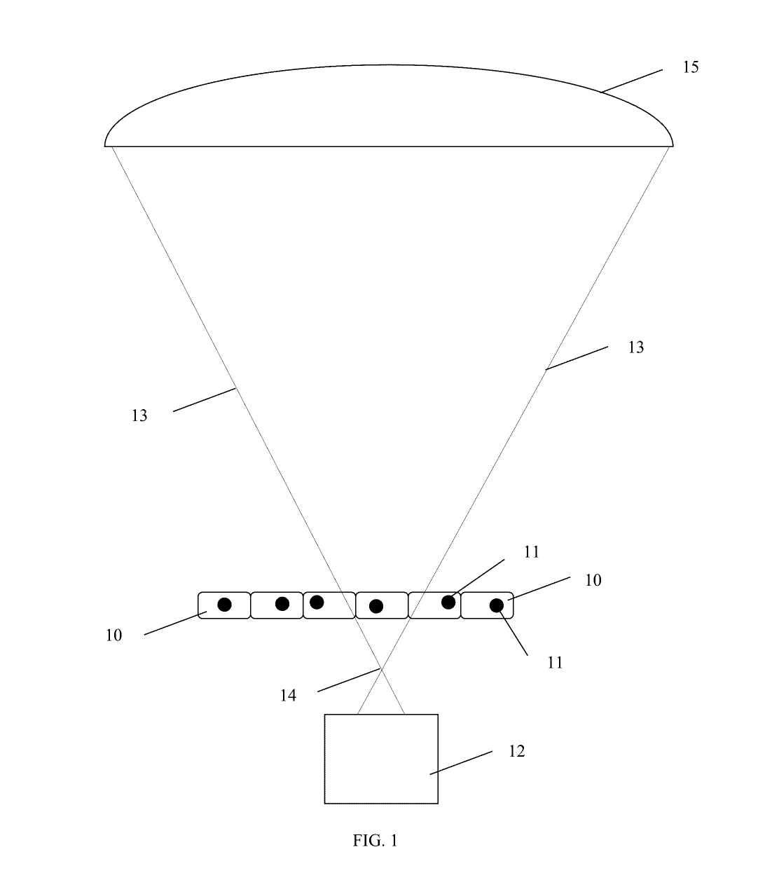

FIG. 2 is a block diagram of the molecular imaging instrument imaging cell(s) using a diverging beam of photons or particles originated from a small focus or vertex to magnify and image cell(s).

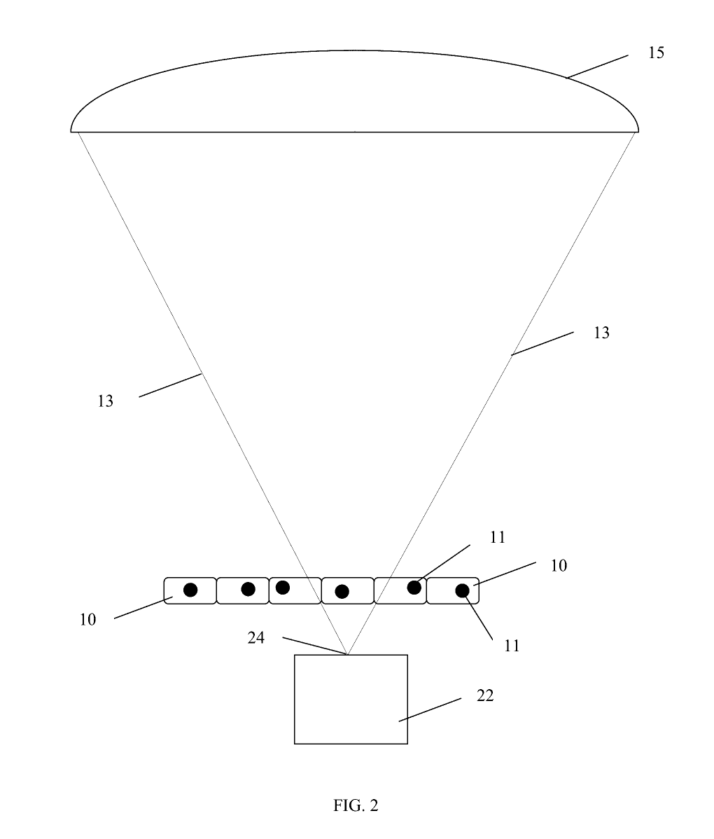

FIG. 3 is a block diagram of the molecular imaging instrument imaging cell(s) using a parallel beam of photons or particles originated from a generator then goes through the cell(s). A device 35 diverges the beams to magnify and image cell(s).

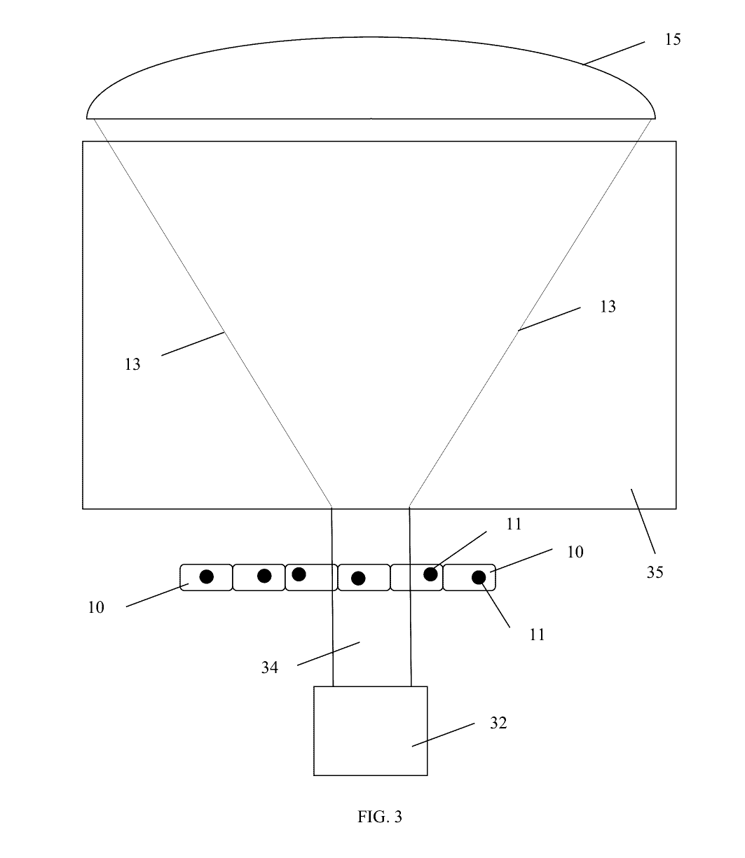

FIG. 4 is a block diagram of the molecular imaging instrument imaging cell(s) using a photons or particles emitted from the chemicals inside the cell. A device magnifies and focuses the photons or particles onto detector to be imaged.

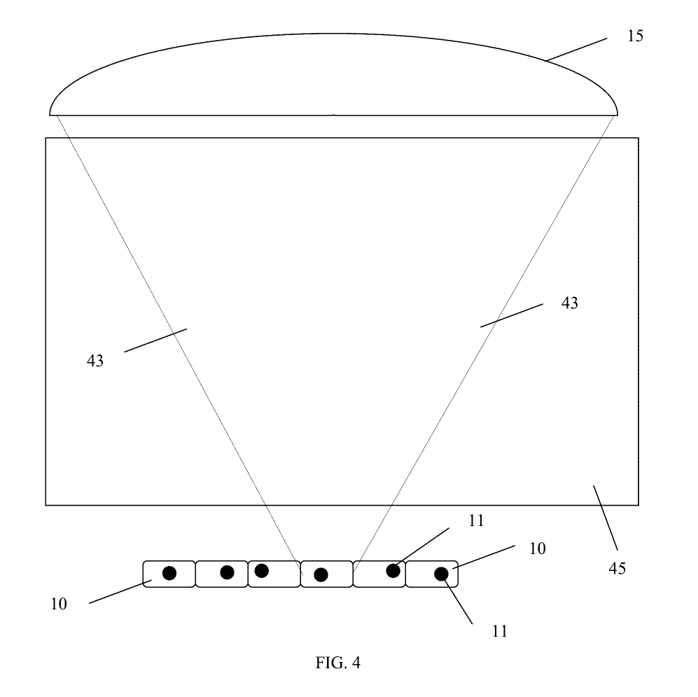

FIG. 5 is a diagram of a solid state pixel detector showing the pixel array, the guard ring and the alignment marks.

FIG. 6 is a diagram of a solid state or scintillation pixel detector showing the detector on top, the readout integrated circuit (IC) at the bottom and the pixels in between. The detector can be connected to the IC in different ways such as indium bump bonds, conductive epoxy, metal wires, and direct contact.

FIG. 7 is a drawing of a two-dimensional (2D0 array of pixel detectors to form large area imaging devices. The top image shows a three-dimensional drawing of the whole array and the bottom drawing shows a cross section showing how the pixel detectors aligned.

FIG. 8 is a schematic diagram of the detector readout electronics circuitry for the input charge sensitive and/or transconductance amplifier placed inside each pixel on the readout IC.

FIG. 9 is a diagram of the small, approximately cell size, pixel detector placed on top of a cell under investigation by imaging radiopharmaceuticals placed into the cell or tagged onto molecules inside the cell.

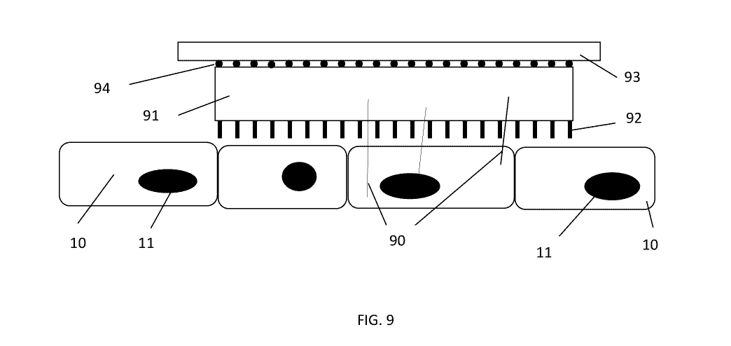

### BEST MODE FOR CARRYING OUT THE INVENTION

Best embodiment: Molecular imaging instrument imaging cell(s) 10 with nuclei 11. Generator 12 produces a conic or fan beam 13 of photons 13 and/or particles 13. The beam is focused into a small vertex 14 and then diverges from the focus point 14. The beam then passes through the cell(s) 10 and is imaged by the detector 15.

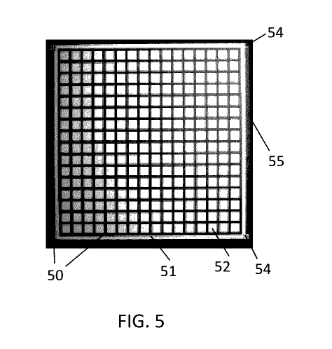

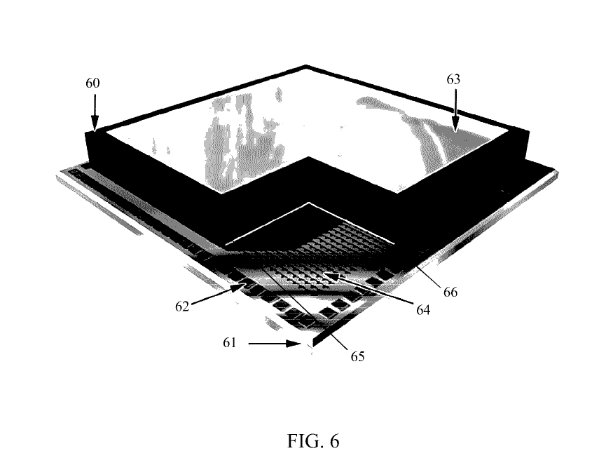

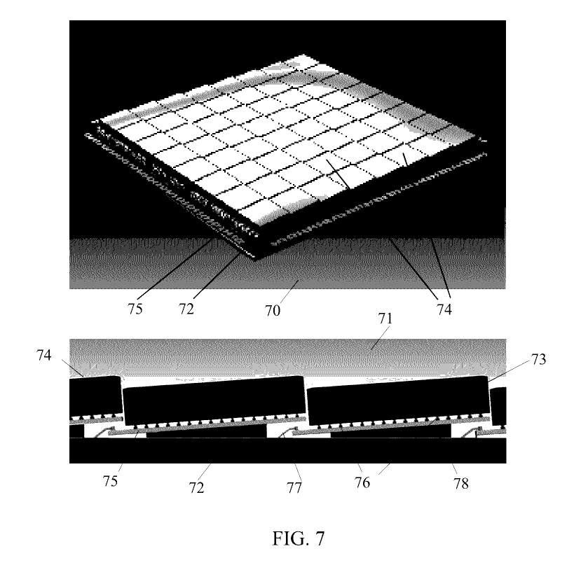

The detector can be made of different embodiments as shown in FIGS. 5-7. The first embodiment of the detector is one pixel detector (FIGS. 5 & 6) or an array of pixel detectors (FIG. 7). FIG. 5 shows fabricated CdZnTe solid state detectors 50. These CdZnTe detectors 50 with pixel electrodes 52 are fabricated and the indium bump 64 bonding is carried out. This process needs high quality solid state detector material such as single or polycrystalline CdZnTe, GaAs, Si, C, Se, Ge, HgI₂ and PbI² material; fabrication of high-quality gold or platinum or other type of electrodes 52 for the pixels 52 and the HV bias pad(s) 63. The indium bump bonding is an important technique for producing low-capacitance, high-quality uniform bonds between detector arrays and ASICs (Application Specific Integrated Circuits). The pixel array consists of 2×2 to 1,000,000×1,000,000 array of 0.001×0.001 to 500×500-micron pitch gold, metal or conductive blocking or non-blocking pixels pads 52 or electrodes (FIG. 6).

A guard ring 51 is also fabricated around the periphery of the pixel array to protect pixels from edge effects and allow also a more uniform response throughout the two-dimensional array. The guard ring is also connected to the readout IC or ASIC using one or more indium bumps. This will allow the biasing of the guard ring with respect to the pixels. For example, the guard ring 51 can be biased to ground, or any other negative or positive voltage, which ever produces the best results. The biasing of the guard ring 51 is done through the IC or ASIC by external circuitry. Other novel guard ring structures can be also designed such as a grid type guard ring. After the crystals had been prepared, rectangular or other geometric forms of indium bumps were deposited both on the detector material/crystal readout pads and on the corresponding ASIC pads. Using alignment marks 54, the two were then aligned on top of each other, the pixilated indium bump sides facing each other, and pressed together to fuse the bumps, a process which takes place at room temperature. If necessary, an underfill 66 of insulating epoxy can be used between the ASIC and the CdZnTe to provide additional support and provide a more robust assembly. It is also possible to epoxy the sides or just the corners. In practice, with a large number of small pixels, this is not usually necessary. In other embodiments detector material can be deposited directly on to the IC in crystal or amorphous form or the detector crystals can be grown directly on the IC in single, multi crystal or amorphous forms.

FIG. 6 show a concept drawing of the hybrid pixel detector and its structure. It shows the pixelated solid state detector such as CdZnTe detector 60. On its top is the gold or platinum HV bias electrode 63. Under the solid state detector there are pixel electrodes 65 made from metal such as gold or platinum. The pixilated readout ASIC 61 is shown under the solid state detector. The detector 60 and ASIC 61 have identical pixel size and geometry so that they will match when bonded together. Normally both the detector and ASIC pixels have indium bumps 64 on them. The detector pixel array and the ASIC are aligned and pressed together so that the indium bumps join and produce the contact between the detector pixel and the ASIC input circuit. Solder and other bonding systems such an asymmetric conductive medium may also be used to produce a contact between the detector pixel and the ASIC pixel input. The electron-hole pairs produce by an x-ray photon moves to the electrodes (holes to cathode and electrons to anode) under the HV Bias and detected and recorded by the ASIC. The ASIC also have contact pads 62 on the perimeter, one, two, three or four sides, so that it can be connected to external circuitry, control system, power supplies, ground, I/O, etc. The detector and ASIC may have all shapes, physical dimensions, thicknesses, sizes, array dimensions, pixel pitch, pixel geometry, etc. depending on the application.

The pixel detectors can be made abutable on one, two or three sides to facilitate tiling to form larger arrays. This means that all the I/O and power pads must be on three, two and one side of the ASIC, respectively. For example, if the connection pads are on two adjacent sides, then 4 sensor arrays can be abutted to each other using the two adjacent sides with no connection pads to form an array with effectively 4 times the active area. An array with all the connection pads are on one side of the ASIC can be abutted to form a uniform array of any size as shown in FIG. 7 where on top it shows a 3D view of the whole detector array 70 and at the bottom a cross section of the array 71. Where the individual pixel detectors 74 are mounted as shown at the bottom section of the figure onto a printed circuit board (PCB) 72. The solid state detector such as CdZnTe 73 is indium bump 76 bonded onto the ASIC 75. The ASIC is wire bonded 77 onto the PCB 72. The ASICs rest on wedge shaped supports under them 78 so that they clear the ASIC connection pads and the wire bonds 77 of the ASIC behind them. The HV bias is applied to the top surface 74.

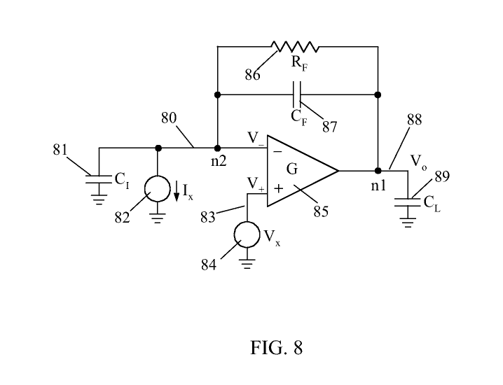

A charge pulse from the detector (equivalent circuit 81 & 82 is given in FIG. 8) goes to an amplifier 85 in FIG. 8 in each pixel of the detector. The amplifier 85 can be all types, such as charge sensitive or transconductance type. The positive input 83 of the amplifier fed a voltage source 84. The amplifier has a feedback circuit made from a capacitor 87 and/or resister 86. The noise and linearity specification for this charge-sensitive amplifier is or is not very stringent. In the later case, in fact, sufficient performance can be achieved using a single transistor amplifier. The complete circuit is shown in FIG. 8. The output 88 goes to a load 89.

A pixel detector FIG. 6 or the pixel detector array FIG. 7 can be used for the imaging detector 15. Other position sensitive detectors and/or position sensitive photomultiplier tubes, CCD arrays can also be used for the detector 15. Instead of detector microscopes of all kinds can be used, such as the optical microscope, scanning microscope and the electron microscope.

### MODE(S) FOR CARRYING OUT THE INVENTION

### Second Embodiment

Molecular imaging instrument imaging cell(s) 10 with possible nuclei 11. Generator 22 produces a conic or fan beam 13 of photons 13 and/or particles 13. The beam is generated from a small vertex 24 and diverged from the source point 24. The beam passes through the cell(s) 10 and is imaged by the detector 15. The detector 15 can be formed as discussed above.

### Third Embodiment

Molecular imaging instrument imaging cell(s) 10 using a parallel beam 34 of photons 34 or particles 34 originated from a generator 32 then goes through the cell(s) 10. A device 35 diverges the beams to magnify and image cell(s) on the detector 15. The detector 15 can be formed as discussed above.

### Fourth Embodiment

Molecular imaging instrument imaging cell(s) using photons 43 or particles 43 emitted from the radiochemicals or radiopharmaceutical inside the cell. A device 45 magnifies and/or focusses the photons or particles onto detector 15 to be imaged. The detector 15 can be formed as discussed above.

### INDUSTRIAL APPLICABILITY

There are many industrial applications for the described invention. Most of these are listed above in the Specifications section. In medical applications the proposed technology and methods can be used to cure, prevent or control diseases and develop new drugs by communicating with cells. In the industrial sector new bio instruments and apparatus can be made with built in intelligence.

What is claimed:

1. An imaging system for detecting the intelligence of at least one cell or eukaryote, the system comprising:
at least one radioactive chemical administered to at least one virus;
the at least one virus genetically programmed;
at least one probe comprising the at least one genetically programmed virus;
a system for administering the at least one probe to the at least one cell or eukaryote;
the at least one probe injects at least one of its DNA into the at least one cell or eukaryote;
a biological activity is created by the at least one DNA of the at least one probe inside the at least one cell or eukaryote;
at least one pixel detector placed at proximity to the at least one cell or eukaryote;
wherein a plurality of particles are emitted from the at least one radioactive chemical during the biological activity and emerge from the at least one cell or eukaryote;
wherein a portion of the plurality of particles enter into the at least one pixel detector;
wherein a portion of the plurality of particles that enter the at least one pixel detector are detected and a plurality of signals are produced;
wherein a processor system processes the plurality of signals and produces at least one image of the biological activity in the at least one cell or eukaryote; and
wherein the at least one image of the biological activity is displayed and at least one information is learned on the intelligence of at least one cell or eukaryote.

2. The system of claim 1, wherein the at least one probe further comprising at least one prokaryote treated with at least one radioactive chemical.

3. The system of claim 1, wherein the at least one cell or eukaryote further comprising at least one prokaryote.

4. The system of claim 1, wherein the information provides knowledge on the physical or the biological structure of the at least one cell or eukaryote.

5. The system of claim 1, wherein the at least one probe establishes communication with the at least one other cell or eukaryote.

6. The system of claim 1, wherein the communication affects biological or cellular activity of the at least one cell or eukaryote.

7. The system of claim 1, wherein the biological activity modifies at least one biological or cellular process, or at least one biological or cellular function in the at least one cell or eukaryote.

8. The system of claim 1, wherein the biological activity inform the at least one cell or eukaryote to start to communicate with a plurality of other cells or eukaryotes.

9. The system of claim 1, wherein the biological activity causes the at least one cell or eukaryote to develop nanotechnology based nanostructures or bionanotechnology based bionanostructures.

10. A method of imaging for detecting the intelligence of at least one cell or eukaryote, the method comprising:
administering at least one radioactive material to at least one virus;
genetically programming the at least one virus;
creating at least one probe comprising the at least one genetically programmed virus;
administering the at least one probe to the at least one cell or eukaryote;
injecting at least one DNA of the at least one probe into the at least one cell or eukaryote;
producing a biological activity by the at least one DNA of the at least one probe in the at least one cell or eukaryote;
placing at least one pixel detector at proximity to the at least one cell or eukaryote;
emitting a plurality of particles from the at least one radioactive chemical involved in the biological activity, which emerge from the at least one cell or eukaryote;
entering into the at least one pixel detector, a portion of the plurality of particles are detected and produce a plurality of signals;
processing at least one portion of the plurality of signals by a processor system and producing at least one image of the biological activity in the at least one cell or eukaryote; and
displaying the at least one image of the biological activity and obtaining at least one information on the intelligence of at least one cell or eukaryote.

11. The method of claim 10, wherein the at least one probe further comprising at least one prokaryote treated with at least one radioactive chemical.

12. The method of claim 10, wherein the at least one cell or eukaryote further comprising at least one prokaryote.

13. The method of claim 10, wherein the information provides knowledge on the physical or the biological structure of the at least one cell or eukaryote.

14. The method of claim 10, wherein the communication disrupts biological or cellular activity of the at least one cell or eukaryote.

15. A method of imaging for detecting the intelligence of at least one cell or eukaryote through communication, the method comprising:
administering at least one radioactive material to at least one virus;
genetically programming the at least one virus;
creating at least one probe comprising the at least one genetically programmed virus;
administering the at least one probe to the at least one cell or eukaryote;
opening at least one door into the membrane of the at least one cell or eukaryote by the at least one probe;
injecting at least one DNA of the at least one probe into the at least one cell or eukaryote;
producing a biological activity by the at least one DNA of the at least one probe in the at least one cell or eukaryote;
placing at least one pixel detector in proximity of the at least one cell or eukaryote;
detecting at least one portion of the radioactivity produced in the biological activity;
imaging the biological activity and producing at least one image;
interpreting the at least one image of the biological activity;
communicating with the at least one cell or eukaryote using the interpretation of the at least one image of the biological activity and the genetic programming of the virus; and
learning at least one information on the intelligence of the at least one cell or eukaryote through the communication.

16. The method of claim 15, further comprising: curing at least one disease of the at least one cell or eukaryote as a result of the biological activity.

17. The method of claim 15, further comprising: genetically programming the at least one DNA of the at least one cell or eukaryote using the least one DNA injected by the at least one probe.

18. The method of claim 15, wherein the biological activity producing at least one enzyme, at least one protein, at least one chemical, at least one virus, at least one, prokaryote, at least one new cell or at least one new eukaryote.

19. The method of claim 15, wherein the at least one cell or eukaryote establishes communication with at least one other cell or eukaryote as a result of the communication.

20. The method of claim 15, further comprising: accelerating biological or cellular evolution of the at least one cell or eukaryote in response to the communication.
